(19) United States
(12) Patent Application Publication
(10) Pub. No.: US 2026/0272432 A1
(43) Pub. Date: Sep. 17, 2026

(54) DYNAMIC AND/OR REPOSITIONABLE TISSUE TRACTION DEVICES, SYSTEMS, AND METHODS

(71) Applicant: BOSTON SCIENTIFIC SCIMED, INC., Maple Grove, MN (US)

(72) Inventors: Scott Edward Corbeil, Litchfield, NH (US); Barry D. Weitzner, Acton, MA (US); Gonzalo Jose Saenz Villalobos, Alajuela (CR)

(73) Assignee: BOSTON SCIENTIFIC SCIMED, INC., Maple Grove, MN (US)

(21) Appl. No.: 19/565,014

(22) Filed: Mar. 12, 2026

Related U.S. Application Data
(60) Provisional application No. 63/771,428, filed on Mar. 13, 2025.

(51) Int. Cl. A61B 17/00; A61B 1/018; A61B 17/28

(52) U.S. Cl. CPC A61B 17/00234; A61B 1/018; A61B 17/282; A61B 2017/00238; A61B 2017/00296; A61B 2017/00367

(57) ABSTRACT

A tissue traction system allowing dynamic application of traction and/or repositionable “pulley” points for application of traction to tissue at a treatment site. The traction may be applied by an elongate element extending from a tissue-lifting tissue-engagement element engaged with tissue at the treatment site, to a traction-adjusting tissue-engagement element engaged with tissue at a force vector-changing location spaced apart from the target tissue, and out of the patient for application of force to the elongate element from outside the patient. The position of the traction-adjusting tissue-engagement element may be adjusted within the patient's body to adjust the traction force vector applied along the elongate element to the tissue-lifting tissue-engagement element.

### CROSS-REFERENCE TO RELATED APPLICATIONS

This application claims the benefit of priority under 35 U.S.C. § 119 to U.S. Provisional Application No. 63/771,428, filed on Mar. 13, 2025, the entire disclosure of which is hereby incorporated by reference herein for all purposes.

### FIELD

The present disclosure relates generally to the field of medical devices, systems, and methods for applying traction to anatomical tissue.

### BACKGROUND

Endoscopic Mucosal Resection (EMR) is an endoscopic procedure which involves the placement of a snare around tissue and either contracting the snare to transect (cold), or using electrosurgical current to transect (hot) the encircled tissue. Endoscopic Submucosal Dissection (ESD) is an endoscopic procedure, first developed in Japan in the late 1990's, used to remove superficial gastrointestinal neoplasms via en bloc resection as a reaction to the shortcomings of EMR. Typically, ESD procedures are technically more challenging than EMR procedures, with longer procedural times and higher rates of complications. However, en bloc resection allows good histopathological assessment of lesion margins, lower recurrence rates, and a greater minimally invasive potential for cure.

Minimally-invasive (e.g., transluminal, transcatheter, endoscopic, etc.) surgical techniques like ESD and EMR typically allow for faster recovery than with open or laparoscopic surgical procedures. However, because such procedures are minimally invasive, there is limited space to maneuver within the body, and such procedures typically require a high degree of expertise. In procedures involving cutting of tissue, one of the largest time and complexity drivers is managing the tissue being cut. The loose section of tissue may obstruct visibility, such as by falling on the instruments being used, occluding visibility of the imaging device (e.g., camera), and creating a hindrance affecting movement of the instruments used during the procedure and in reaching all regions and depths of the target tissue being cut. Various solutions for lifting the cut (and often hanging) mass of tissue, thus clearing the path for visibility and operation of medical tools and devices, have been developed. However, positioning of the elements used with such solutions may be challenging, particularly in a space-restricted environment. Moreover, as tissue is cut, a continual increase in traction is required, presenting various challenges during the procedure. Additionally, the devices used with such solutions may require separate medical tools than those used to perform the procedure, and such tools may even require a separate working channel, thereby potentially increasing the size and/or complexity of the delivery system. As endoscopic submucosal dissection continues to evolve with new techniques, and new instruments are designed for making the procedure faster and safer, there remains a need for improvements to application of traction to tissue during the ESD procedure. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In some aspects, a tissue traction system is formed in accordance with various principles of the present disclosure for transluminal delivery into a patient's body to apply traction to tissue at a treatment site within the patient's body. In some aspects, the tissue traction system includes a tissue-lifting tissue-engagement element; a traction-adjusting tissue-engagement element; and an elongate element having a distal end coupled to the tissue-lifting tissue-engagement element, and extending from the tissue-lifting tissue-engagement element to be operably associated with the traction-adjusting tissue-engagement element for relative movement with respect thereto, the elongate element extending proximally and transluminally to a proximal end controllable from outside the patient. In some aspects, traction applied to the tissue-lifting tissue-engagement element by the elongate element is adjustable within the patient's body by pulling on the elongate element from outside the patient to move the elongate element with respect to the traction-adjusting tissue-engagement element as a pulley to apply traction to the tissue-lifting tissue-engagement element.

In some aspects, the elongate element extends from the tissue-lifting tissue-engagement element, through a portion of the traction-adjusting tissue-engagement element, and proximally to the proximal end outside the patient's body. In some aspects, the traction-adjusting tissue-engagement element defines an aperture therethrough and the elongate element extends through the aperture. In some aspects, the traction-adjusting tissue-engagement element is operably coupled to an elongate member defining a lumen therethrough, the elongate element extending through the lumen to outside the patient's body.

In some aspects, the tissue traction system further includes a tubular elongate member defining a lumen through which the tissue-lifting tissue-engagement element and the traction-adjusting tissue-engagement element are delivered. In some aspects, the tubular elongate member is delivered with a medical delivery device defining a working channel through which a medical instrument is deliverable into the patient. In some aspects, the tubular elongate member is laterally separable from the medical delivery device to allow independent movement of the medical delivery device while the traction-adjusting tissue-engagement element is engaged with tissue.

In some aspects, the tissue traction system further includes an elongate control element having a distal end coupled to the tissue-lifting tissue-engagement element and operable to engage and deploy the tissue-lifting tissue-engagement element with tissue. In some aspects, the elongate control element is rotatable to rotationally engage the tissue-lifting tissue-engagement element with tissue, and is frangibly coupled with the tissue-lifting tissue-engagement element to be separated therefrom after the tissue-lifting tissue-engagement element is deployed with respect to tissue.

In some aspects, the traction-adjusting tissue-engagement element is engageable with the tissue-lifting tissue-engagement element and movable to engage and deploy the tissue-lifting tissue-engagement element with tissue.

In some aspects, the traction-adjusting tissue-engagement element is configured to be engageable with tissue spaced apart from the treatment site, releasable from the engaged tissue, and movable to another location spaced apart from the treatment site and engaged with tissue at the other location.

In some aspects, a tissue traction system is formed in accordance with various principles of the present disclosure for transluminal delivery into a patient's body to apply traction to tissue at a treatment site within the patient's body. In some aspects, the tissue traction system includes a tissue-lifting tissue-engagement element; a traction-adjusting tissue-engagement element; an elongate element having a first end coupled to the tissue-lifting tissue-engagement element, and extending from the tissue-lifting tissue-engagement element to be operably associated with the traction-adjusting tissue-engagement; and an elongate member operably coupled with the traction-adjusting tissue-engagement element to move the traction-adjusting tissue-engagement element to be engaged with tissue at a first location, to move the traction-adjusting tissue-engagement element from the first location to a second location, and to move the traction-adjusting tissue-engagement element to be engaged with tissue at the second location to adjust the traction force vector applied by the elongate element to the tissue at the treatment site via the tissue-lifting tissue-engagement element.

In some aspects, the elongate element is operably associated with the traction-adjusting tissue-engagement element to be translatable with respect to the traction-adjusting tissue-engagement element to adjust the traction force vector applied by the elongate element to the tissue-lifting tissue-engagement element.

In some aspects, the traction-adjusting tissue-engagement element includes a pair of jaws shiftable between a tissue-receiving configuration in which the jaws are spaced apart to receive tissue therebetween or to release tissue, and a tissue-grasping configuration in which the jaws grasp tissue therebetween, the jaws being controllable from outside the patient's body to engage the traction-adjusting tissue-engagement element with tissue at the first location, to release tissue at the first location, and to engage tissue at the second location. In some aspects, the tissue-lifting tissue-engagement element is helical and the jaws of the traction-adjusting tissue-engagement element are engageable with the tissue-lifting tissue-engagement element to rotationally engage the tissue-lifting tissue-engagement element with tissue to engage tissue; and the tissue-lifting tissue-engagement element includes an extension graspable by the jaws of the traction-adjusting tissue-engagement element to rotationally advance the tissue-lifting tissue-engagement element into tissue.

In some aspects, a method of applying traction to tissue within a patient includes, in accordance with various principles of the present disclosure, transluminally advancing a tissue-lifting tissue-engagement element and a traction-adjusting tissue-engagement element with an elongate element extending therebetween to a treatment site within a patient; engaging the tissue-lifting tissue-engagement element with tissue at the treatment site; engaging the traction-adjusting tissue-engagement element with tissue at a first location spaced apart from the treatment site; extending the elongate element from the tissue-lifting tissue-engagement element to the traction-adjusting tissue-engagement element and proximally to outside the patient; and proximally pulling on the elongate element from outside the patient to apply traction to tissue at the treatment site via the elongate element and the tissue-lifting tissue-engagement element.

In some aspects, the method further includes moving the elongate element with respect to the traction-adjusting tissue-engagement element to adjust the traction force vector along the elongate element. In some aspects, the method further includes extending the elongate element from the tissue-lifting tissue-engagement element to through an aperture defined through the traction-adjusting tissue-engagement element and then proximally out of the patient's body. In some aspects, the method further includes advancing the traction-adjusting tissue-engagement element into the patient coupled to a distal end of an elongate member, and extending the elongate element from the tissue-lifting tissue-engagement element and into a lumen defined through the elongate member and proximally out of the patient's body. In some aspects, the method further includes disengaging the traction-adjusting tissue-engagement element from the first location, moving the traction-adjusting tissue-engagement element to a second location spaced apart from the target tissue, and engaging the traction-adjusting tissue-engagement element with tissue at the second location to adjust the traction force vector applied to the elongate element by pulling on the elongate element from outside the patient.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a tissue traction system formed in accordance with aspects of the present disclosure.

FIGS. 2A-2H illustrate delivery and deployment and use of an example of an embodiment of a tissue traction system formed in accordance with various principles of the present disclosure.

FIG. 3 illustrates a perspective view of an example of an embodiment of a tissue-lifting tissue-engagement element, a traction-adjusting tissue-engagement element, and an elongate element of a tissue traction system formed in accordance with various principles of the present disclosure.

FIG. 4A and FIG. 4B illustrate delivery and deployment of an example of an embodiment of a tissue traction system formed in accordance with various principles of the present disclosure.

FIGS. 5A-5C illustrate delivery and deployment and use of an example of an embodiment of a tissue traction system formed in accordance with various principles of the present disclosure.

FIG. 6A and FIG. 6B illustrate delivery and deployment of an example of an embodiment of a tissue traction system formed in accordance with various principles of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

There has been a growing interest in the medical field in minimally-invasive procedures, e.g., transcatheter, endoscopic, laparoscopic, etc., which do not require open surgery (cutting open the patient), but, instead, access a target site within the patient via a natural orifice (or, in some instances, a small incision not considered to constitute an open-surgery cut). For the sake of convenience, and without intent to limit, such procedures may be broadly referenced herein as transluminal procedures, in contrast with surgical procedures utilizing large surgical openings made in the patient for generally direct access (e.g., without navigating through a device or internal body passage) to the site at which a procedure is to be performed (e.g., target tissue). Various endoluminal surgical (ELS) procedures have been developed, such as, without limitation, Endoscopic Mucosal Resection (EMR), Endoscopic Submucosal Dissection (ESD), to be performed within a patient and not in direct view of the medical professionals performing the procedure. The lack of direct visibility, as well as typical limited space for maneuvering of medical instruments presents various challenges to the medical professionals performing the procedure.

Although EMR can be performed in piecemeal technique, pathology on tissue fragments is often challenging and patients require more frequent follow-up endoscopies due to higher rate of incomplete resections and recurrences. While EMR is associated with relatively low technical complexity, shorter procedure times, and a low risk of adverse events, en bloc EMR is limited typically to lesions which are less than 2.0 cm in diameter/width. More specifically, for large sessile or laterally spreading polyps, en bloc resection with convention EMR is nearly impossible for lesions greater than 2 cm. For colorectal lesions of greater than 2 cm, en bloc resection can only be achieved in about 30% of cases.

Medical professionals thus often turn to ESD for resection of challenging lesions, as well as large lesions. It is not uncommon for ESD to be utilized to remove neoplasms between 2-7 cm. The European Society of Gastrointestinal Endoscopy (ESGE) recommends ESD be used first line in the removal of Gastric Superficial neoplastic lesions with very low risk of lymph node metastases (EMR may be considered if the lesion is less than 10 to 15 mm with a very low probability of advanced histology). Often, ESD is also recommended first line for superficial esophageal squamous cell cancer (with EMR an option for lesions which are less than 10 mm). For colonic and rectal lesions, ESGE recommends removal of lesions with polypectomy or EMR. However, ESD can be considered if there is a high suspicion of limited submucosal invasion (indicated by depressed morphology and an irregular or non-granular surface pattern), and especially if the lesion is greater than 20 mm, or in lesions that otherwise cannot be optimally and radically removed by snare-based techniques (such as EMR techniques).

Although ESD has been in practice for two decades, ESD remains a very technically challenging procedure. In Japan, where ESD procedure is best established, the complication rates (including perforation, peritonitis, and bleeding) are 3.5% for gastric ESD, 3.3% for esophageal ESD, and 4.6% for Colorectal ESD. Outside of Japan and China, EDS is practiced by a relatively small number of highly technically-skilled endoscopists and surgeons. Long procedure times and high risk of perforation are still significant challenges which have prevented broad physician adoption.

In accordance with various principles of the present disclosure, devices, systems, and methods are described which may be used in minimally-invasive procedures, such as ESD, to facilitate manipulation of an anatomical structure within the patient's body. The anatomical structure may be anatomical tissue, and the device may be configured to move a portion or layer of tissue relative to another portion or layer of tissue. It will be appreciated that reference may be made herein simply to “tissue” instead of “anatomical tissue” or “anatomical structure” for the sake of convenience and without intent to limit. In some aspects, the present disclosure is directed to devices, systems, and methods for moving, such as to separate and/or lift and/or apply traction to tissue within a patient's body.

Traction is a foundational technique utilized in open and laparoscopic surgery. Traction may be used to move tissue or other anatomical structures and may be advantageously used to greatly enhance visualization of anatomy, tissue planes, and potential hazards such as nerves or blood vessels. Additionally or alternatively, traction may be used to impart a tension force to the tissue, which may change the way the tissue behaves as it is being cut or dissected. For instance, traction may be used in surgical procedures to “present” the tissue to be cut to the cutting instrument in an orientation or manner that makes the cutting easier and/or helps to insure that the cut is made through the desired tissue in the desired direction. In some aspects, traction alters the nature and/or properties of tissue in a manner which may facilitate performance of a procedure with respect to or in the vicinity of the tissue under traction. It will be appreciated that principles of the present disclosure are applicable to tissue which is cut as well as tissue or other anatomical structures which are not cut. For instance, principles of the present disclosure are applicable for providing traction and/or counter-traction during various medical procedures, including, without limitation, surgical procedures that involve cutting of tissue. The use of traction is a basic precept of surgery during cutting procedures, but should be appreciated as having broader applicability as well.

In addition to the procedural benefits of using traction during endoscopic procedures, there is a mounting body of evidence that devices aiding with traction during endoscopic submucosal dissection reduce the technical complexity of the procedure, and significantly lower the learning curve associated with trainees and/or new ESD procedure adopters.

To date, current traction solutions, both commercialized and physician made, do not adequately address true user needs. At best, commercially-available traction solutions leave significant room for improvement with regard to efficiency, effectiveness, flexibility, and ease of use. In open surgery, traction typically is applied using the non-dominant hand directly or through a surgical instrument (typically some kind of forceps). In laparoscopic surgery, traction is applied via tissue graspers introduced through one of the surgical ports, which provide access to the target body cavity or space. This can be done by the lead surgeon, assisting surgeon, or scrub nurse. In endoscopic surgery, however, multiple portals for accessing the target surgical site generally are not provide, and, typically, only the endoscope (or other access device) provides both visualization and access. Although there are some endoscopes with two working channels, or devices that can attach to the outside of the endoscope to provide a secondary working channel, all of these access channels are either integral to the endoscope or run parallel to it, and therefore are directionally constrained, and limited in the degrees of freedom of movement and control that can be achieved when maneuvering other devices to the target site.

In accordance with various principles of the present disclosure, dynamic and/or repositionable traction devices, systems, and methods with which an elongate element may be operably coupled with tissue at a traction target site within a patient's body, and traction applied to the tissue via the elongate element. It will be appreciated that the elongate element may be any elongate element suitable for transmitting traction to tissue (directly or via another element), such as, without limitation, a tether, filament, string, cord, wire, band, loop, dental floss, etc., either elastic or inelastic, stretchable or not stretchable, metallic or polymeric or combination thereof, etc., and typically sufficiently flexible to be navigated through curved passages (e.g., within the patient). It will be appreciated that reference may be made to a treatment site, anatomical site, delivery site, deployment site, implant/implantation site, site of implantation, target site, etc., interchangeably and without intent to limit. In some aspects, target tissue is defined within the treatment site and with respect to which a procedure is to be performed. The target tissue may be, but is not necessarily limited to, a lesion. In some aspects, the elongate element is operably coupled with a tissue-engagement element configured to engage tissue at the treatment site (e.g., to engage target tissue). The tissue-engagement element may be in the form of a clip, clamp, or other device with structures movable (such as, without limitation, pivotable) with respect to each other to grasp tissue therebetween with or without penetrating the tissue (e.g., a device with two or more jaws movable between a tissue-receiving configuration in which the jaws are spaced apart to receive tissue therebetween, and a tissue-grasping configuration in which the jaws are moved together to grasp tissue therebetween); and/or a tissue-penetrating device such as a device with tissue-penetrating helix, barbs, hooks, tines, etc., capable of penetrating tissue to retain the tissue-engagement element with respect to tissue; and/or other configurations known to those of ordinary skill in the art, the present disclosure not being limited in this regard. It will be appreciated that terms such as engage (and other grammatical forms thereof) may be used interchangeably herein with terms such as, without limitation, couple, grasp, hold, clasp, clip, clamp, anchor, attach, affix, secure, etc. (and other grammatical forms thereof), without intent to limit. The tissue-engagement element is configured to be securely engaged with tissue to transmit to the tissue traction applied by the elongate element. In some aspects, an additional tissue-engagement element is provided to operably associate the elongate element with another location within the patient's body to modify at least the direction of the traction force vector applied by the elongate element to the tissue at the treatment site (e.g., the target tissue). It will be appreciated that reference herein to engagement of a tissue-engagement element with tissue is intended as secure, anchoring engagement which resists withdrawal therefrom, even upon application of traction force thereto. In some aspects, the tissue-engagement elements may be considered tissue anchors. In some aspects, the tissue-engagement elements are delivered together. In some aspects, at least one of the tissue-engagement elements is configured to engage another one of the tissue-engagement elements to assist with deployment of the other one of the tissue-engagement elements.

In some aspects, the magnitude of the traction force vector, the direction of the traction force vector, or both the magnitude and the direction of the traction force vector applied by a tissue traction system formed in accordance with various principles of the present disclosure are adjustable. In some aspects, the elongate element is movable with respect to the additional tissue-engagement element to modify the magnitude of the force vector to allow for dynamic control of traction during the procedure. For instance, in some aspects, the elongate element is fixedly secured with respect to the tissue-engagement element which is engaged with tissue at the treatment site, and movable (e.g., slidable, translatable, etc.) with respect to the additional tissue-engagement element engaged with tissue spaced apart from the treatment site. Additionally or alternatively, the location at which the additional tissue-engagement element engages tissue may be changed during the procedure to allow modification of the position of the additional tissue-engagement element during the procedure, thereby allowing dynamic control of traction during the procedure in this manner (instead of or in addition to the above-described dynamic traction control). Such adjustment of the location of the additional tissue-engagement element may allow for adjustment of the magnitude and/or direction of the traction force applied by an elongate element operably coupled with target tissue and operably associated with the additional tissue-engagement element.

In some aspects, a tissue traction device or system formed in accordance with various principles of the present disclosure is configured for delivery and use and removal (withdrawal) in a minimally invasive manner. Thus, in contrast with delivery, use, and removal through a surgically formed opening through which the patient's body is substantially directly accessed, a tissue traction device and/or system of the present disclosure may be delivered, operated, and/or removed with a delivery device which extends into the patient through a natural orifice and/or a small incision. For instance, a tissue traction device and/or system of the present disclosure may be delivered, operated, and/or removed through a tubular elongate member (such as an endoscope, a laparoscope, a catheter, a sheath, a tube, etc.) which is inserted into the patient's body through a natural orifice (e.g., natural body opening such as the mouth) and/or a small incision (not large enough for direct access to the target site within the patient's body). Additionally or alternatively, a tissue traction device and/or system of the present disclosure may be delivered, operated, and/or removed substantially directly (e.g., without a surrounding nonanatomic lumen) into a body lumen, passage, etc., of the patient for delivery, through such body lumen, passage, etc., to a target site. For the sake of convenience, reference is made herein to transluminal delivery to incorporate minimally invasive procedures including, but not limited to, procedures such as described above, which access the body through a natural orifice (which may be known as an “incisionless” procedure) or through a small incision (which may be known as a percutaneous procedure), to advance a device or system into the patient's body to a target site, such as via a lumen (of the body or of a delivery device), rather than through a large surgically-created opening which would provide generally direct access to a target site. In some aspects, a tissue traction device or system formed in accordance with various principles of the present disclosure is configured for insertion into a patient's body alongside (e.g., along the exterior) of a medical instrument delivery device configured to deliver medical instruments to the treatment site for use in performing a procedure with respect to the treatment site. It will be appreciated that terms such as medical instruments, tools, devices, accessories, etc. may be used interchangeably herein without intent to limit. The delivery device may define a lumen therethrough. In some aspects, the delivery device may be an endoscope with a flexible elongate member (also known as an insertion tube) configured for insertion into a patient's body and defining a working channel therethrough through which medical instruments are deliverable into a patient's body. In some aspects, the tissue-engagement element which engages tissue at the treatment site and/or the tissue-engagement element which engages tissue spaced apart from the treatment site are delivered in an over-the-scope manner (alongside/along the exterior of a medical delivery device). In some aspects, the tissue-engagement elements are delivered through a tubular elongate member operably associated with the medical delivery device. In some aspects, the elongate element extends through the tubular elongate member from the tissue-engagement elements to a proximal end for control (e.g., application of traction force thereto) by a medical professional. In some aspects, the tubular elongate member is separable from the medical delivery device to allow independent movement of the medical delivery device while the tubular elongate member is positioned adjacent to tissue to which a tissue-engagement element, delivered by the tubular elongate member, is operably engaged.

Various embodiments of devices, systems, and methods for applying traction to tissue will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features in the drawings are identified herein and in the drawings by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). Moreover, certain features in one embodiment may be used across different embodiments and are not necessarily individually labeled when appearing in different embodiments.

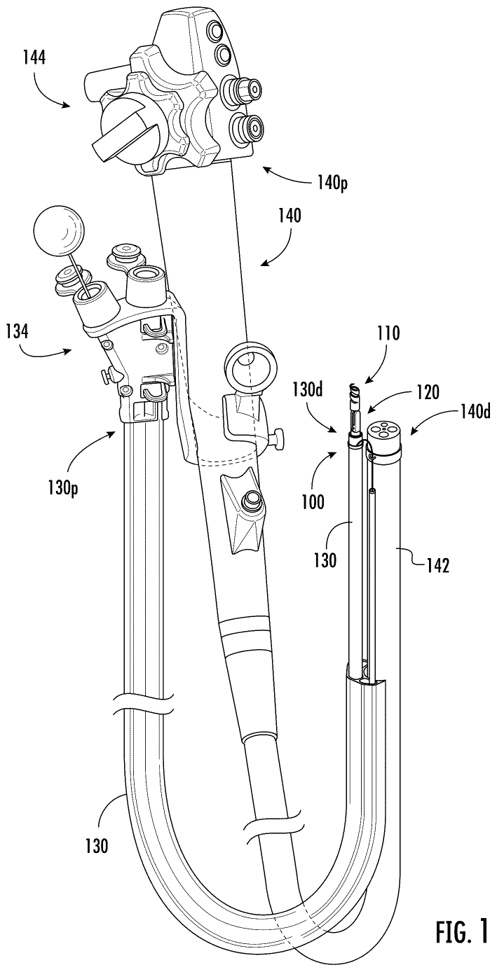

Turning now to the drawings, an example of an embodiment of a tissue traction system 100 formed in accordance with various principles of the present disclosure is illustrated in FIG. 1. The tissue traction system 100 includes at least two tissue-engagement elements 110, 120, each configured to be securely engaged with tissue in the vicinity of a treatment site within a patient's body so that traction may be applied via the tissue-engagement elements 110, 120 to tissue to perform a procedure with respect to the treatment site. The tissue-engagement elements 110, 120 of a tissue traction system formed in accordance with various principles of the present disclosure are in any form known to those of ordinary skill in the art which is capable of engaging anatomical tissue. For instance, tissue-engagement elements of the present disclosure may be in the form of two or more graspers (e.g., two or more jaws movable with respect to each other between a tissue-receiving configuration in which the jaws are spaced apart to receive tissue therebetween, and a tissue-grasping configuration in which the jaws are moved together to grasp tissue therebetween); a helical member (e.g., laser-cut tube, helical wire, screw, etc.) capable of being rotated into tissue to be secured with respect to the tissue); and/or other tissue-penetrating element (e.g., barbs—either retractable non-retractable, hooks, tines, etc.) sized, shaped, configured, dimensioned, and/or formed of a suitable material capable of penetrating tissue to retain the tissue-engagement element with respect to tissue; a suction element (e.g., utilizing a vacuum source to hold tissue in place with respect to the element); and/or other configuration known to those of ordinary skill in the art, the present disclosure not being limited in this regard.

In some aspects, the tissue-engagement elements 110, 120 are delivered via a delivery device in the form of a tubular elongate member 130. In some aspects, the tubular elongate member 130 is delivered along with (e.g., alongside, such as along the exterior of) a medical delivery device 140 defining a lumen 141 through which other medical tools, instruments, devices, accessories, etc. (such terms being usable interchangeably herein without intent to limit) may be delivered to the treatment site to perform a procedure. In some aspects, the tubular elongate member 130 is operably associated with the delivery device 140 such as for delivery therewith into a patient. As such, the tubular elongate member 130 may be considered to provide an auxiliary lumen for the tissue traction system 100 in addition to the lumen 141 of the delivery device 140. The delivery device 140 may be any suitable device known to those of ordinary skill in the art for delivering a medical instrument. In FIG. 1, the delivery device 140 is illustrated as an endoscope, but may be any other device, such as a tubular elongate member such as a catheter, overtube, sheath, etc. In the illustrated example of an embodiment, an insertion tube 142 extends along the distal end 140d of the delivery device 140 and is configured for insertion and navigation within the patient's body. The tubular elongate member 130 may extend along the exterior of the insertion tube 142 of the delivery device 140. In some aspects, at least a portion of the tubular elongate member 130 and/or the delivery device 140 which is inserted into the patient's body is sufficiently flexible to be navigated into and within a patient (e.g., through curved and/or tortuous body passages within the patient) to a treatment site. In some aspects, the tubular elongate member 130 and/or the delivery device 140 may be considered to be an element of the tissue traction system 100. The delivery device 140 may include a control handle 144 at a proximal end 140p thereof to control movement and/or navigation of the insertion tube 142. Additionally or alternatively, a control handle 134 may be provided along the proximal end 130p of the tubular elongate member 130 to control one or more elements of the tissue traction system 100.

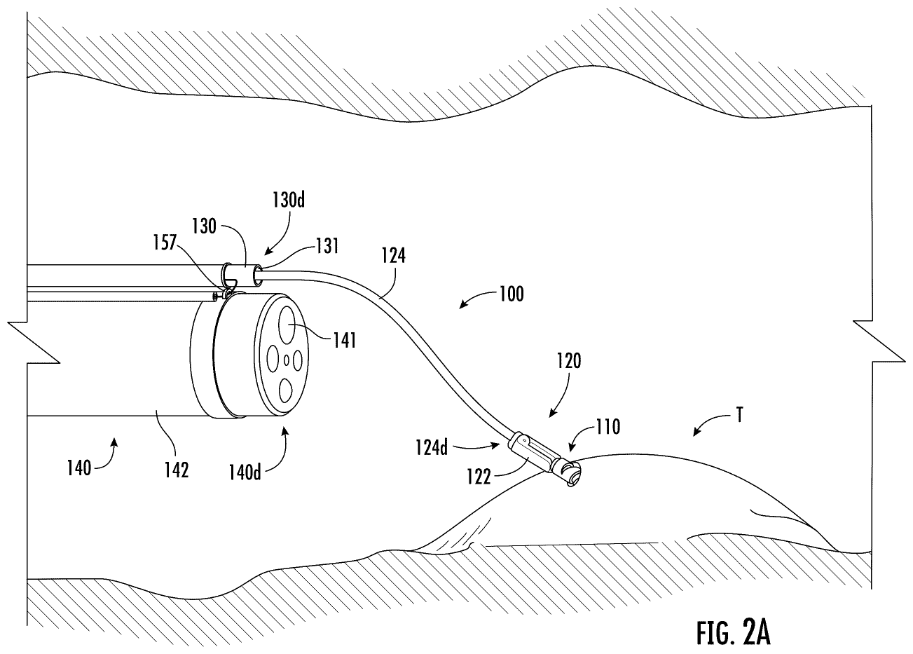

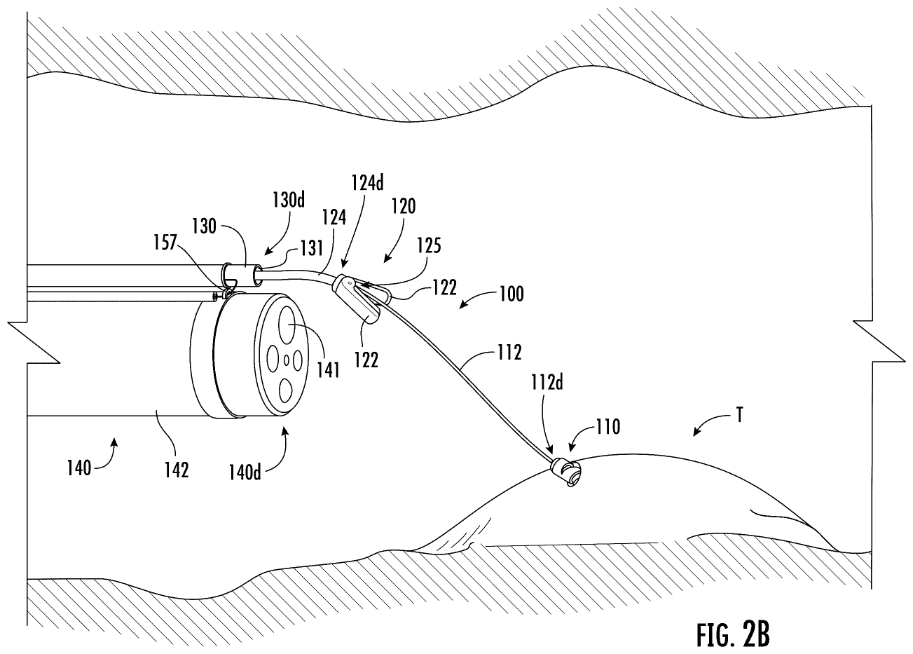

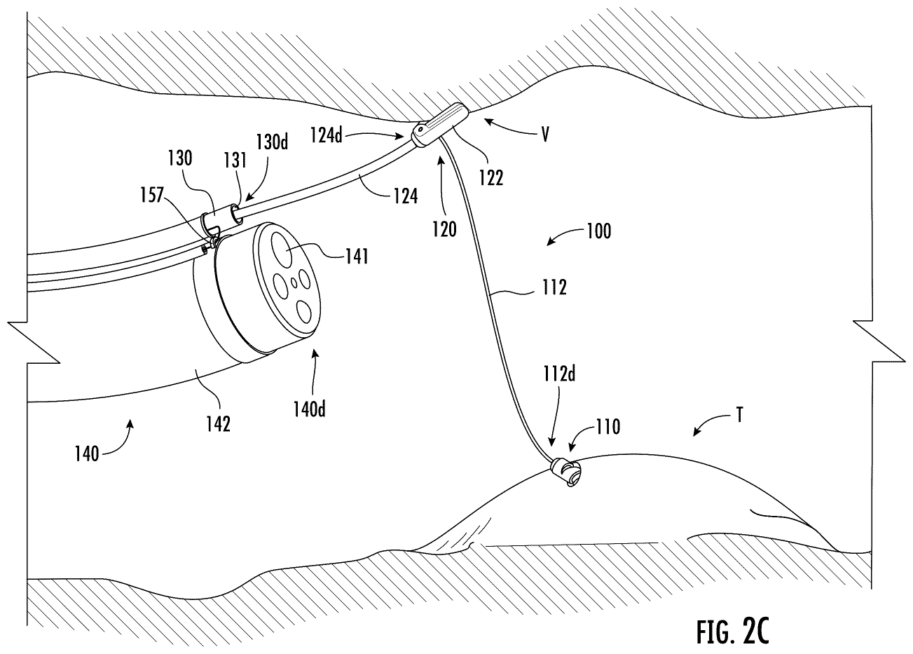

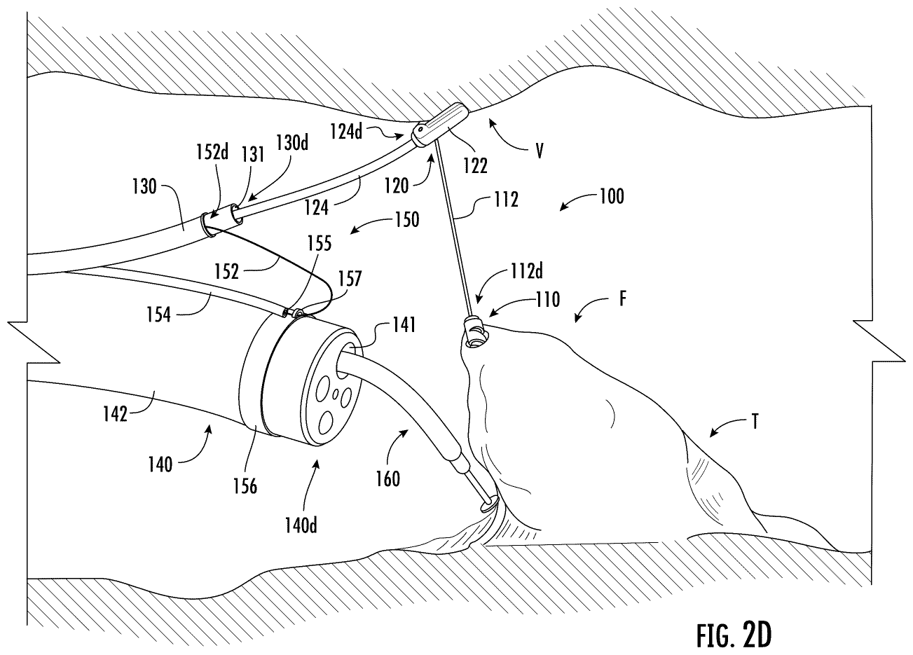

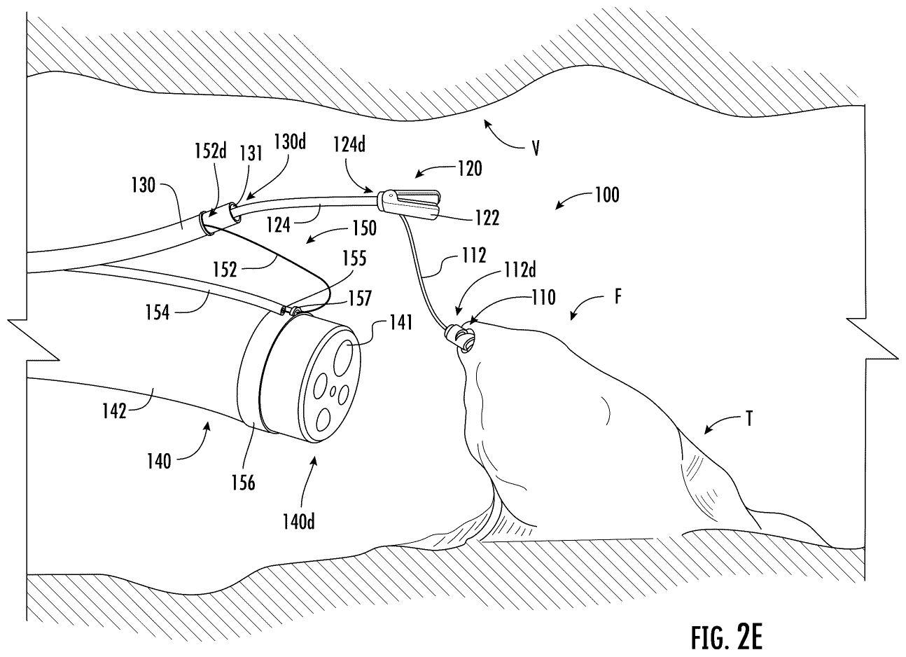

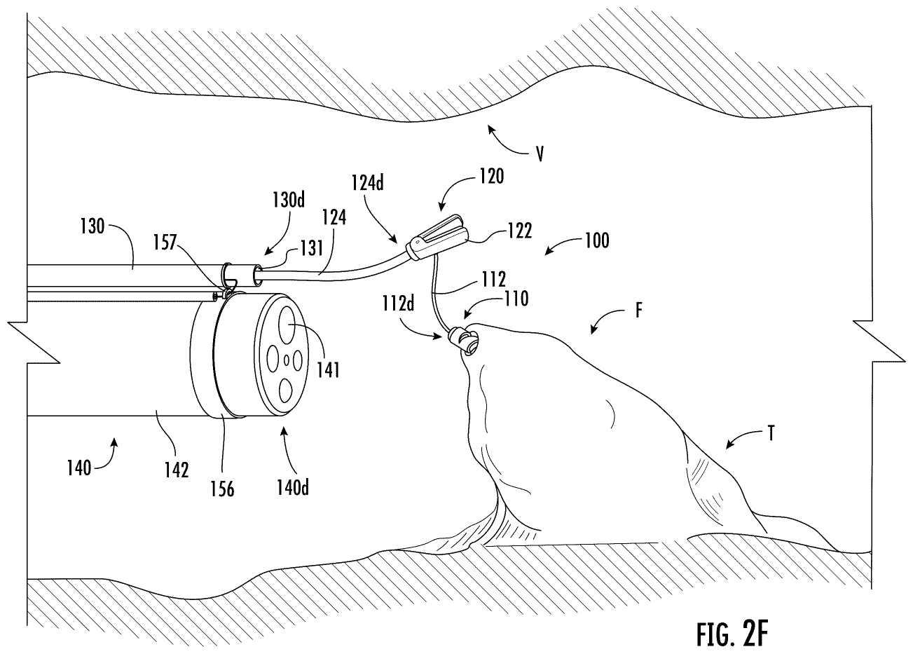

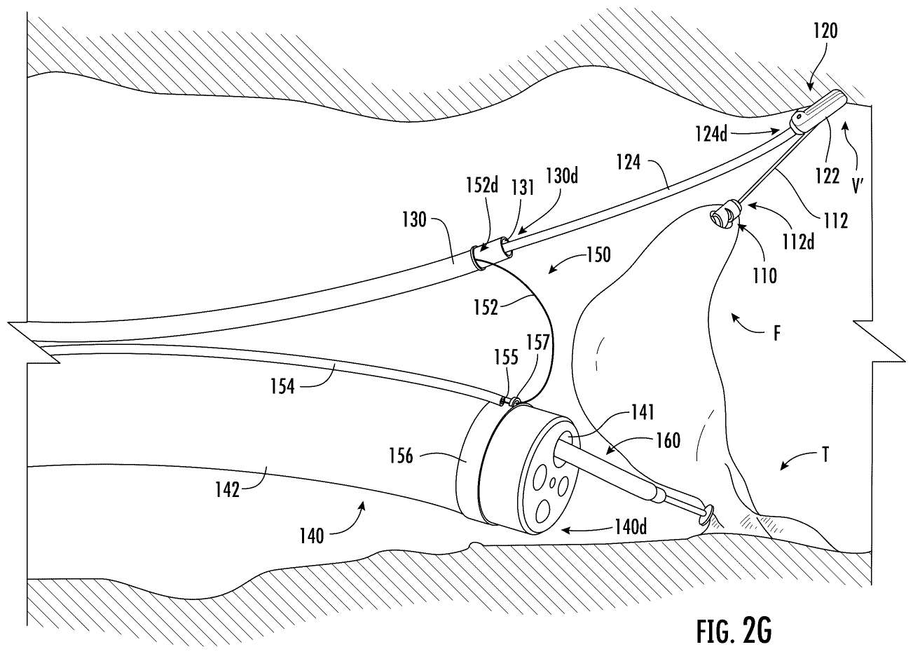

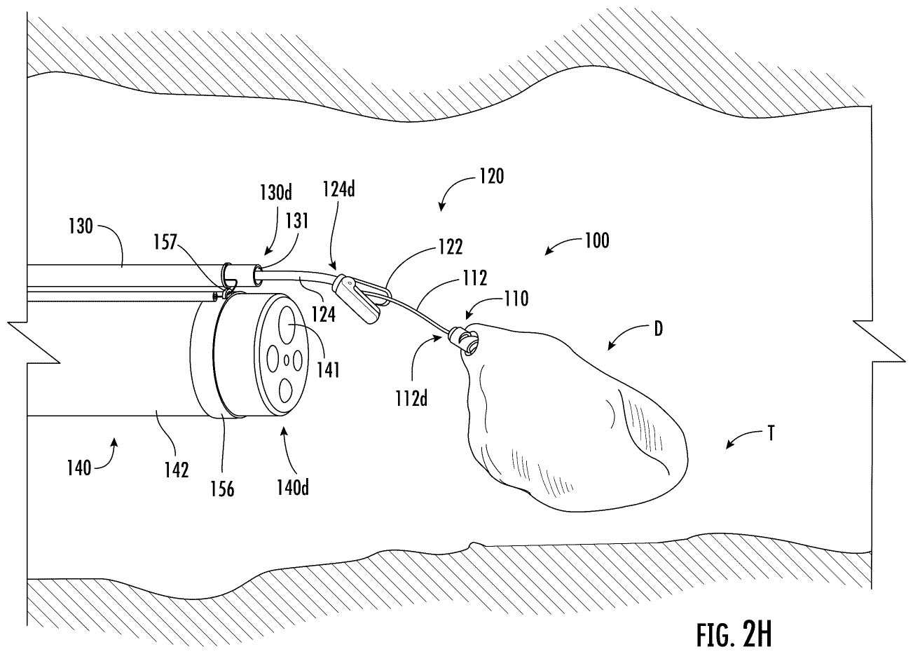

As may be appreciated with reference to FIGS. 2A-2H, the at least two tissue-engagement elements 110, 120 of the tissue traction system 100 illustrated in FIG. 1 are operably associated with one another to allow separate, spaced apart deployment to achieve the desired and/or medically indicated force vectors on tissue at a treatment site T. More particularly, as illustrated in FIGS. 2A-2H, at least one tissue-engagement element 110 of the tissue traction system 100 is engaged with tissue along the treatment site T. Another tissue-engagement element 120 of the tissue traction system 100 is engageable with tissue at a location spaced apart from the treatment site T and the tissue-engagement element 110, such as illustrated in FIG. 2C, FIG. 2D, and FIG. 2G, with a traction-transmitting element 112 (not visible in FIG. 2A, but illustrated in FIGS. 2B-2H) operably associated with the tissue-engagement elements 110, 120. The traction-transmitting element 112 is operably coupled with respect to the tissue-engagement element 110 for application of traction to the treatment site T via the tissue-engagement element 110. The tissue-engagement element 120 is positioned spaced apart from the tissue-engagement element 110 and configured to affect and/or adjust the force vector (magnitude and/or direction) of traction applied by the traction-transmitting element 112 to the tissue-engagement element 110 and thus to tissue with which the tissue-engagement element 110 is engaged.

For the sake of convenience, and without intent to limit, to differentiate the tissue-engagement element 110 which is engaged with tissue at the treatment site T from the tissue-engagement element 120 (including additional tissue-engagement elements) engaged with tissue spaced apart from the treatment site T (e.g., to adjust the force vector of the traction applied to the tissue-engagement element 110 at the treatment site T), the tissue-engagement element 110 engaged with tissue at the treatment site T is referenced herein as a tissue-lifting tissue-engagement element 110, and the tissue-engagement element 120 spaced apart from the treatment site T is referenced herein as a traction-adjusting tissue-engagement element 120. It will be appreciated that additional tissue-engagement elements which are engaged with tissue along the treatment site T may also be referenced as tissue-lifting tissue-engagement elements. It will further be appreciated that additional tissue-engagement elements which are engaged with tissue spaced apart from the treatment site T yet which are operably associated with a tissue-lifting tissue-engagement element to apply traction thereto may also be referenced as traction-adjusting tissue-engagement elements.

In the example of an embodiment illustrated in FIG. 2B, the distal end 112d of the traction-transmitting element 112 is operably associated with the tissue-lifting tissue-engagement element 110. In some aspects, the distal end 112d of the traction-transmitting element 112 is fixedly coupled with respect to the tissue-lifting tissue-engagement element 110. The distal end 112d of the traction-transmitting element 112 may be secured to the tissue-lifting tissue-engagement element 110 in any of a variety of manners known to those of ordinary skill in the art, such as by being operably associated with an opening through the tissue-lifting tissue-engagement element 110 or an eyelet or other coupling mounted with respect to the tissue-lifting tissue-engagement element 110 (e.g., looped through the opening or with respect to the coupling and knotted, inserted through the opening and crimped or otherwise increased in size to resist withdrawal, etc.); welded; adhered, etc. In some aspects, the distal end 112d of the traction-transmitting element 112 may be coupled with the tissue-lifting tissue-engagement element 110 such that rotation of the tissue-lifting tissue-engagement element 110 to become engaged with tissue does not cause entanglement of the traction-transmitting element 112. A proximal end of the traction-transmitting element 112 extends proximally for operable control by a medical professional to apply force thereto. In some aspects, the proximal end of the traction-transmitting element 112 extends outside the patient's body, or is operably associated with an element (e.g., controller) extending or positioned outside the patient's body and accessible by a medical professional to apply and adjust a force with respect to the traction-transmitting element 112 to transmit to the tissue-engagement element 110, as described in further detail below. The tissue-lifting tissue-engagement element 110 is configured to be securely engaged with respect to tissue at the treatment site T to convey or transmit traction forces applied by the traction-transmitting element 112 to the tissue at the treatment site T, such as to lift the tissue as it is being cut, as described in further detail below. Accordingly, the engagement of the tissue-engagement element 110 with tissue is preferably sufficiently secure to withstand application of traction forces thereto via the traction-transmitting 112 without becoming dislodged or separated from the treatment site T.

In the example of an embodiment of a tissue traction system 100 illustrated in FIGS. 2A-2H, the traction-transmitting element 112 is in the form of an elongate element 112 and is thus referenced heretofore as such, without intent to limit. The elongate element 112 may be any elongate element known to those of ordinary skill in the art for applying traction to tissue, such as a filament, traction band, an elastic band, a cord, a cable, a loop, a suture, nitinol spring steel, a wire (e.g., high carbon spring wire, music wire, muscle wire, etc.), dental floss, and/or any other suitable elongate member. Typically, the elongate element 112 is sufficiently flexible to be navigated within the tubular elongate member 130 to the treatment site T. The elongate element 112 may be metallic, polymeric, elastomeric, or a combination of metallic, polymeric, and/or elastomeric. In some aspects, the elongate element 112 is elastic. The elongate element 112 may be stretchable (e.g., elongatable) or non-stretchable, the present disclosure not being limited in this regard.

The tissue traction system 100 illustrated in FIGS. 2A-2H may be used to apply traction to tissue at a treatment site T as follows. As illustrated in FIG. 2A, the tissue traction system 100 is delivered via the tubular elongate member 130 to the treatment site T. For instance, the illustrated example of an embodiment of a tubular elongate member 130 defines a lumen 131 through which the tissue-engagement elements 110, 120 may be delivered to a treatment site within a patient. In some aspects, the tissue-engagement elements 110, 120 are delivered distal to the lumen 131 of the tubular elongate member 130, such as illustrated in FIG. 1. Optionally, the tissue-engagement elements 110, 120 may be delivered to the treatment site T within the lumen 131 of the tubular elongate member 130 and then distally advanced out of the tubular elongate member 130.

The example of an embodiment of a tissue-lifting tissue-engagement element 110 of the tissue traction system 100 of the present disclosure is illustrated as a helical tissue-engagement element and may be securely engaged with tissue at the treatment site T by being rotated into the tissue. The example of an embodiment of a traction-adjusting tissue-engagement element 120 of the tissue traction system 100 of the present disclosure is illustrated as having a pair of tissue-engaging jaws 122, which are movable with respect to each other between a tissue-receiving configuration in which the jaws are spaced apart to receive tissue therebetween and a tissue-grasping configuration in which the jaws are moved together to grasp tissue therebetween. However, other configurations of tissue-engagement elements 110, 120, engaged with tissue at the treatment site T in any appropriate manner as known to those of ordinary skill in the art, are within the scope and spirit of the present disclosure. The traction-adjusting tissue-engagement element 120 may be operably coupled with the distal end 124d of an elongate member 124 which may be used to deliver the traction-adjusting tissue-engagement element 120 to the treatment site T. In some aspects, the elongate member 124 is used to move and/or control movement of the traction-adjusting tissue-engagement element 120 with respect to the treatment site T and may be considered a control element, as discussed in further detail below. The elongate member 124 may be sufficiently flexible to be navigated within the tubular elongate member 130 to the treatment site T. In some aspects, the elongate member 124 extends proximally for control by a medical professional. For instance, the elongate member 124 may extend proximally to a proximal end positioned outside the patient's body for control by a medical professional or to another control element manipulable by a medical professional to control the elongate member 124 (e.g., to control movement, placement, deployment, positioning, etc., of the traction-adjusting tissue-engagement element 120 via the elongate member 124). As may be appreciated with reference to FIG. 2B, the elongate element 112 may be substantially coaxially delivered with the elongate member 124, such as through a lumen 125 defined through the elongate member 124. However, other configurations are within the scope and spirit of the present disclosure. For instance, in some aspects, the elongate element 112 may extend alongside the elongate member 124 and through the lumen 131 of the tubular elongate member 130. In other aspects, the elongate element 112 may extend outside and generally along the exterior of the tubular elongate member 130. The present disclosure is not to be limited in this regard. Regardless of the manner in which the elongate element 112 extends proximally for manipulation by a medical professional, in some aspects, the elongate element 112 is operably coupled with the traction-adjusting tissue-engagement element 120 so that the traction-adjusting tissue-engagement element 120 may adjust the traction force vector on the elongate element 112, as described in further detail below.

In the example of an embodiment of a tissue traction system 100 illustrated in FIG. 2A, the traction-adjusting tissue-engagement element 120 is illustrated as delivered with and adjacent to (proximal to) the tissue-lifting tissue-engagement element 110 as the tissue-lifting tissue-engagement element 110 is being deployed and engaged with tissue. In some aspects, the traction-adjusting tissue-engagement element 120 is operably associated with the tissue-lifting tissue-engagement element 110 during deployment of the tissue-engagement element 110. Optionally, such operable association allows the traction-adjusting tissue-engagement element 120 to assist with deployment of the tissue-lifting tissue-engagement element 110, as described in further detail below. However, in other embodiments, the tissue-lifting tissue-engagement element 110 may be deployed separately from the traction-adjusting tissue-engagement element 120 and other tissue-engagement elements of the tissue traction system 100, with other tissue-engagement elements being delivered separately and/or after deployment of the tissue-lifting tissue-engagement element 110, such as in a manner described in further detail below.

Once the tissue-lifting tissue-engagement element 110 has been engaged and deployed with respect to tissue at the treatment site T, such as illustrated in FIG. 2B, the other tissue-engagement element(s) of the traction system 100 may be delivered and deployed to apply and achieve the desired traction on tissue at the treatment site T. As illustrated in FIG. 2B, the traction-adjusting tissue-engagement element 120 is withdrawn from the tissue-lifting tissue-engagement element 110 after the tissue-lifting tissue-engagement element 110 is engaged with tissue. The tubular elongate member 130, and optionally also the delivery device 140, may then be moved to deploy the traction-adjusting tissue-engagement element 120, as illustrated in FIG. 2C, at an appropriate vector-changing location V at which the force vector of the elongate element 112 may be adjusted to apply appropriate traction force to the tissue-lifting tissue-engagement element 110. The details of the manner in which the traction-adjusting tissue-engagement element 120 is deployed and engaged with tissue at the vector-changing location V are not illustrated, but may be readily appreciated by those of ordinary skill in the art. For instance, the elongate member 124 may be advanced distally from the tubular elongate member 130, the jaws 122 actuated from a closed configuration (in which the jaws are adjacent one another, such as illustrated in FIG. 2A), in which the jaws 122 are typically moved within the patient's body (e.g., to be in a low profile), to a tissue-receiving configuration (illustrated, for instance, in FIG. 2B) to engage tissue at the vector-changing location V between the jaws 122, and then the jaws 122 may be returned to the configuration grasping tissue such as illustrated in FIG. 2C. In some aspects, a control element (e.g., control wire) such as known in the art extends proximally from the jaws 122 for actuation by medical professional. In some aspects, the control element extends proximally along or through the elongate member 124.

As may be appreciated with reference to FIG. 2C, the elongate element 112 of the illustrated example of an embodiment of a tissue traction system 100 extends from the tissue-lifting tissue-engagement element 110 to the traction-adjusting tissue-engagement element 120 and then proximally for control by a medical professional (e.g., to adjust traction applied thereto). In accordance with various principles of the present disclosure, the elongate element 112 is operably associated with the traction-adjusting tissue-engagement element 120 to move (e.g., slide) with respect to the traction-adjusting tissue-engagement element 120 while the traction-adjusting tissue-engagement element 120 and the tissue-lifting tissue-engagement element 110 are engaged with tissue. For instance, the elongate member 124 from which the traction-adjusting tissue-engagement element 120 extends may define a conduit or lumen 125 (see, e.g., FIG. 2B) through which the elongate element 112 may extend proximally for control by a medical professional (e.g., to a control handle 134, such as illustrated in FIG. 1, or for manual grasping by a medical professional). In some aspects, the proximal end of the elongate element 112 may be operably coupled with a ratcheting reel (e.g., such as known to those of ordinary skill in the art), which may be provided on the control handle 134, to allow the medical professional a fine level of control over the traction force to be applied to the elongate element 112, and optionally to lock a selected tension at a desired level, and to release such tension as needed. In some aspects, the proximal end of the elongate element 112 may be operably coupled with a control reel which may incorporate a drag or over-tensioning feature which would allow the tension on the filament to slip if too much tension is applied, to prevent inadvertent tearing of tissue-lifting tissue-engagement element 110 out of the tissue in which it had been deployed and engaged. In some aspects, a weight may be attached to the proximal end of the elongate element 112 to allow gravity to apply traction force to the elongate element 112. The distal opening of the lumen 125 may serve as a pulley point for the elongate element 112 to adjust the force vector of the traction applied to the elongate element 112 and thus to the tissue-lifting tissue-engagement element 110 and tissue at the treatment site T. The distalmost end of the elongate member 124 from which the elongate element 112 extends may be rounded or otherwise radiused to allow smooth movement (e.g., sliding or axial/longitudinal translation) of the elongate element 112 therealong. As such, the tissue traction system 100 allows dynamic adjustability of the magnitude of the traction force applied to tissue at the treatment site T via the tissue-lifting tissue-engagement element 110 and the elongate element 112.

Once the tissue-engagement elements 110, 120 have been engaged with tissue, or at least once the tissue-lifting tissue-engagement element 110 has been engaged with tissue, a medical instrument 160 may be delivered to the treatment site T to perform a procedure with respect to the treatment site T, such as illustrated in FIG. 2D. More particularly, in the example illustrated in FIG. 2D, the medical instrument is a cutting device (e.g., an electrosurgical knife, although the present disclosure need not be limited in this regard) used to cut tissue at the treatment site T. In some aspects, prior to engagement of the tissue-lifting tissue-engagement element 110 with tissue at the treatment site T, and prior to delivery of the medical instrument 160, a fluid (e.g., water, saline, gel, etc.) is injected into tissue at the treatment site T to elevate/lift the tissue (to create a “bleb”) to facilitate cutting of the tissue to create the initial incision.

In some aspects, the tubular elongate member 130 (or at least a distal section thereof) may be at least laterally separable from the delivery device 140 (or at least the flexible elongate member 142 thereof) to allow increased freedom of movement of the delivery device 140, such as for increased freedom of movement of the medical instrument 160 to facilitate accurate and efficient performance of the procedure with respect to the treatment site T while the tubular elongate member 130 is adjacent the traction-adjusting tissue-engagement element 120 engaged with tissue at a vector-changing location V. For instance, the tissue traction system 100 may include a return-to-home system 150 including a return element 152 operably coupling the tubular elongate member 130 and the delivery device 140, such as illustrated in FIG. 2D. The return element 152 may be slackened to allow the delivery device 140 to move away from the tubular elongate member 130 after the traction-adjusting tissue-engagement element 120 is engaged with tissue at the vector-changing location V so that the delivery device 140 may be returned to the treatment site T to perform a procedure while leaving the tubular elongate member 130, through which the traction-adjusting tissue-engagement element 120 extends, with the traction-adjusting tissue-engagement element 120. In some aspects, the return element 152 may be controlled to maintain a desired tension between the tubular elongate member 130 and the delivery device 140 without impeding movement of the delivery device 140 and thus the medical instrument 160. In some aspects, the return element 152 extends within (e.g., through a working channel of) or along the exterior of the delivery device 140 to a proximal end controlled by a medical professional. In some aspects, a tubular elongate member such as a sheath 154 with a lumen 155 defined therethrough is provided along the delivery device 140 and the return element 152 extends proximally through the lumen 155. In some aspects, the proximal end of the return element 152 may be directly manually grasped by a medical professional. Additionally or alternatively, the return element 152 may be coupled with a controller, such as a component of or along a control handle 134 such as illustrated in FIG. 1, by which tension on the return element 152 may be adjusted. The return element 152 may be an elongate flexible element, such as, without limitation, a filament, a string, a cord, a wire, a tether, etc., coupled in any desired manner, such as, without intent to limit, wrapping, adhering, taping, integrating into a bracket or cap, etc., to the tubular elongate member 130. In the non-limiting example of an embodiment illustrated in FIGS. 2A-2H, the distal end 152d of the return element 152 is wrapped around the tubular elongate member 130. In some aspects, prior to entering the lumen 155 of the sheath 154, the return element 152 may extend movably through an aperture 157 defined with respect to an external coupler 156 mounted with respect to the distal end 140d of the delivery device 140.

As the medical instrument 160 cuts tissue at the treatment site T, a flap of tissue F is formed, as illustrated in FIG. 2D. As the tissue at the treatment site T is continued to be cut, such as during performance of an ESD procedure, the flap of tissue T increases in size and further separates from tissue at the treatment site T. The magnitude of the traction force applied to the target tissue F by the elongate element 112 via the tissue-lifting tissue-engagement element 110 may be increased by proximally pulling on the elongate element 112, such as to maintain substantially continuous traction on the flap of tissue F to continue to lift the flap of tissue F away from the medical instrument 160.

In some aspects, the directional vector of the traction force applied by the elongate element 112 to the flap of tissue F may need to be adjusted in order to more effectively direct the flap of tissue F away from the medical instrument 160 and the delivery device 140. In accordance with various principles of the present disclosure, the traction-adjusting tissue-engagement element 120 is releasable from the vector-changing location V, and may be moved to a different vector-changing location V', such as illustrated in FIGS. 2E-2G. More particularly, the traction-adjusting tissue-engagement element 120 may be disengaged and released from the vector-changing location V, such as by moving the jaws 122 to a tissue-releasing spaced-apart configuration (e.g., an open configuration). In some aspects, the traction-adjusting tissue-engagement element 120 and the tubular elongate member 130 may be drawn closer to the delivery device 140, such as illustrated in FIG. 2E, by proximally pulling on the return element 152. In some aspects, once the tubular elongate member 130 and the elongate member 124 of the traction-adjusting tissue-engagement element 120 have been returned to a position adjacent to the distal end 140d of the delivery device 140 (“returned to home”), such as illustrated in FIG. 2F, movement of the delivery device 140 may move the traction-adjusting tissue-engagement element 120 to another vector-changing location V', at which the traction-adjusting tissue-engagement element 120 may engage tissue, such as illustrated in FIG. 2G. Cutting of tissue may be resumed with the direction of traction force (and optionally also the magnitude of the traction force) adjusted to further lift the flap of tissue F as desired and/or medically indicated. Once the target tissue has been dissected and separated from the treatment site T, such as illustrated in FIG. 2H, the traction-adjusting tissue-engagement element 120 may be released from the vector-changing location V′ and “returned to home” adjacent the delivery device 140, such as with the tubular elongate member 130. In some aspects, the tissue-lifting tissue-engagement element 110 may remain engaged with the dissected tissue D and removed with the delivery device 140 from the patient.

As may be appreciated, movability of the traction-adjusting tissue-engagement element 120 of a tissue traction system 100 of the present disclosure allows for adjustability of the magnitude and/or direction of the traction force vector applied by the elongate element 112 which is operably coupled to target tissue (optionally via a tissue-lifting tissue-engagement element 110) and which is operably associated with the traction-adjusting tissue-engagement element 120. As may further be appreciated, as noted above, in some aspects, the elongate member 124 is used to move the traction-adjusting tissue-engagement element 120, such as to adjust the traction force vector of the elongate element 112 operably associated with the traction-adjusting tissue-engagement element 120. In some aspects, a first terminal end of the elongate element 112 is operably coupled to traction tissue, and a second terminal end, opposite the first terminal end, is operably coupled to the traction-adjusting tissue-engagement element 120. With such arrangement, the elongate element 112 remains within the patient's body without having an end extending proximally out of the patient, and forces on the elongate element 112 (and thus on target tissue to which the elongate element 112 is operably coupled) are controlled by moving the traction-adjusting tissue-engagement element 120, such as after the first terminal end of the elongate element 112 is operably coupled to target tissue. In some aspects, forces on the elongate element 112 are controlled by the elongate member 124 which may be used to control the movement, placement, deployment, positioning, etc., of the traction-adjusting tissue-engagement element 120 and thus of the second terminal end of the elongate element 112. In some aspects, the elongate member 124 is movable, such as to control the traction-adjusting tissue-engagement element 120, via a proximal end of the elongate member 124, which may extend outside the patient's body, or another element extending outside the patient's body for control by a medical professional and operably coupled with the elongate member 124. In some aspects, the elongate member 124 remains coupled with the traction-adjusting tissue-engagement element 120 as a control element thereof. For instance, in some aspects, the elongate member 124 remains coupled with the traction-adjusting tissue-engagement element 120 during deployment and use of a tissue traction system 100 formed in accordance with various principles of the present disclosure, and the traction-adjusting tissue-engagement element 120 is withdrawn with the elongate member 124 from the patient's body. In some aspects, the elongate member 124 remains with the traction-adjusting tissue-engagement element 120 throughout the entire use of the tissue traction system 100.

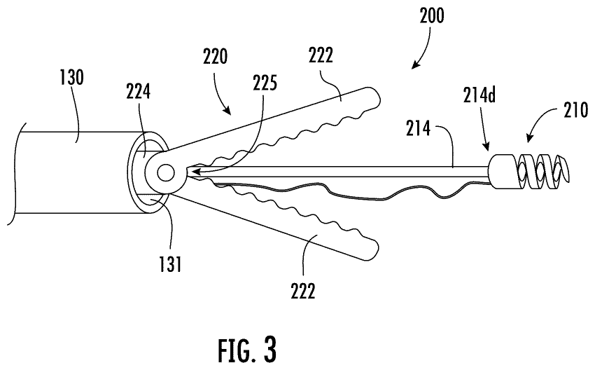

As noted above, the tissue-lifting tissue-engagement element of a tissue traction system formed in accordance with various principles of the present disclosure may be deployed and securely engaged with tissue at the treatment site in any of a variety of manners. In some aspects, an elongate control element 214 is operably coupled with a tissue-lifting tissue-engagement element 210, such as illustrated in FIG. 3. More particularly, the tissue-lifting tissue-engagement element 210 of the example of an embodiment of a tissue traction system 200 illustrated in FIG. 3, is operably coupled with the distal end 214d of an elongate control element 214 operable to engage the tissue-lifting tissue-engagement element 210 with tissue. The illustrated example of an embodiment of a tissue-lifting tissue-engagement element 210 is as a helical tissue anchor, and the elongate control element 214 may be a control wire or stylet or other elongate element capable of transmitting torque along the longitudinal extent thereof (extending through the patient's body, and optionally through the lumen 131 of the tubular elongate member 130) to the tissue-lifting tissue-engagement element 210. In some aspects, the elongate control element 214 extends alongside the elongate member 224 which is operably coupled with the traction-adjusting tissue-engagement element 220. In some aspects, the elongate control element 214 extends through a lumen defined through the elongate member 224. The example of an embodiment of a traction-adjusting tissue-engagement element 220 is illustrated in FIG. 3 as having a pair of jaws 222, although other configurations are within the scope and spirit of the present disclosure.

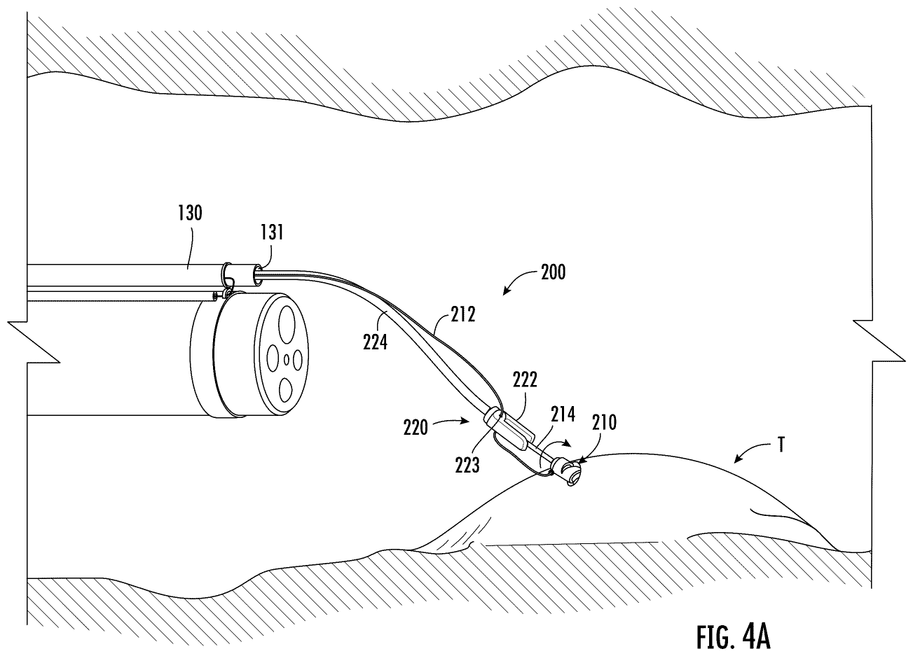

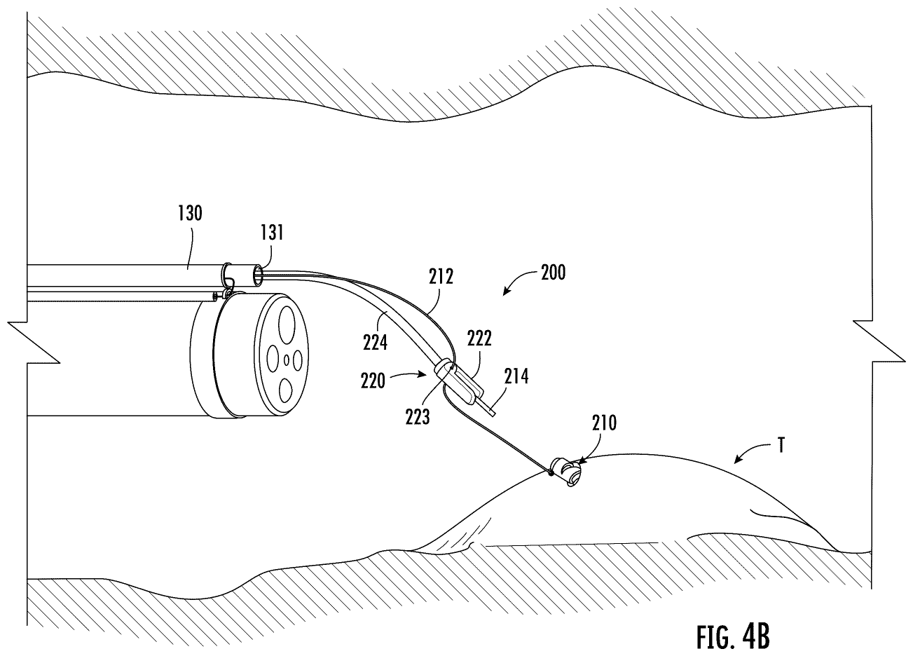

A tissue-lifting tissue-engagement element may be engaged with tissue (to contact and optionally also be secured with respect to tissue) in any of a variety of manners. In the example of an embodiment of a tissue traction system 200 illustrated in FIG. 4A and FIG. 4B, the tissue-lifting tissue-engagement element 210 and the traction-adjusting tissue-engagement element 220 are delivered together to the treatment site T. In some aspects, the tissue-lifting tissue-engagement element 210 may be extended distally spaced from the traction-adjusting tissue-engagement element 220. The elongate control element 214 may be rotated (as schematically illustrated by the arrow in FIG. 4A) to engage and drive/advance (e.g., rotationally) the tissue-lifting tissue-engagement element 110 into tissue at the treatment site T. In some aspects, the elongate element 212 is operably coupled with (e.g., rotationally looped around) the traction-adjusting tissue-engagement element 220 so as not to become entangled by rotation of the traction-adjusting tissue-engagement element 220. For instance, in some aspects, an eyelet or collar with an eyelet, or other appropriate structure may be rotationally coupled with respect to the tissue-lifting tissue-engagement element 210 and/or the elongate member 224, and allow the elongate element 212 to move with respect thereto (e.g., as a pulley point), without becoming tangled, twisted, etc. In some aspects, the elongate control element 214 is removably (e.g., frangibly) coupled with the tissue-lifting tissue-engagement element 210 so that once the tissue-lifting tissue-engagement element 210 is securely engaged with tissue, the elongate control element 214 may be separated from the tissue-lifting tissue-engagement element 210, as illustrated in FIG. 4B. The elongate control element 214 optionally may be proximally withdrawn and removed from the patient's body. The placement of the traction-adjusting tissue-engagement element 220 and performance of the procedure with respect to the treatment site T may as described above with reference to FIGS. 2A-2H, reference being made thereto for the sake of brevity and without intent to limit.

In some aspects, the traction-adjusting tissue-engagement element 220 may be used to rotate the elongate control element 214 to engage and drive/advance (e.g., rotationally) the tissue-lifting tissue-engagement element 210 with tissue. For instance, jaws 222 of a tissue-lifting tissue-engagement element 210 may clamp onto the elongate control element 214, and the elongate member 224 may be rotated to rotate the jaws 222 and thus the elongate control element 214 to rotate the traction-adjusting tissue-engagement element 220 into engagement with tissue. In some aspects, the elongate control element 214 may be advanced along with the tissue-lifting tissue-engagement element 210 to the treatment site T separately from the traction-adjusting tissue-engagement element 220 to engage and drive/advance (e.g., rotationally) the tissue-lifting tissue-engagement element 210 with tissue. The elongate control element 214 may extend through the lumen 225 defined through the elongate member 224 and the elongate member 224 may be advanced to the treatment site T over the elongate control element 214. Once the elongate control element 214 has been separated from the tissue-lifting tissue-engagement element 210, the elongate control element 214 may be withdrawn proximally through the lumen 225 of the elongate member 224. In some aspects, the elongate element 212 extends through the lumen 225 defined through the elongate member 224, such as illustrated in FIG. 3, such as in a manner described above with reference to the lumen 125 through the elongate member 124 described above with reference to FIGS. 2A-2H. For the sake of brevity and without intent to limit, reference is made to such description as applicable mutatis mutandis to an elongate element 212 extending through a lumen 225 such as illustrated in FIG. 3. In some aspects, the elongate element 212 extends through an opening or aperture 223 through the traction-adjusting tissue-engagement element 220, such as illustrated in FIG. 4A and FIG. 4B, so that the traction-adjusting tissue-engagement element 220 may act as a pulley for the elongate element 212, changing the direction of the traction force vector applied to the elongate element 212, the tissue-lifting tissue-engagement element 210, and tissue engaged by the tissue-lifting tissue-engagement element 210. In either arrangement, the proximal end of the elongate element 212 may be extended through the lumen 225 or the aperture 223 associated with the traction-adjusting tissue-engagement element 220 outside the patient's body so that the traction-adjusting tissue-engagement element 220 may be delivered separately from, yet operably associated with, the tissue-lifting tissue-engagement element 210.

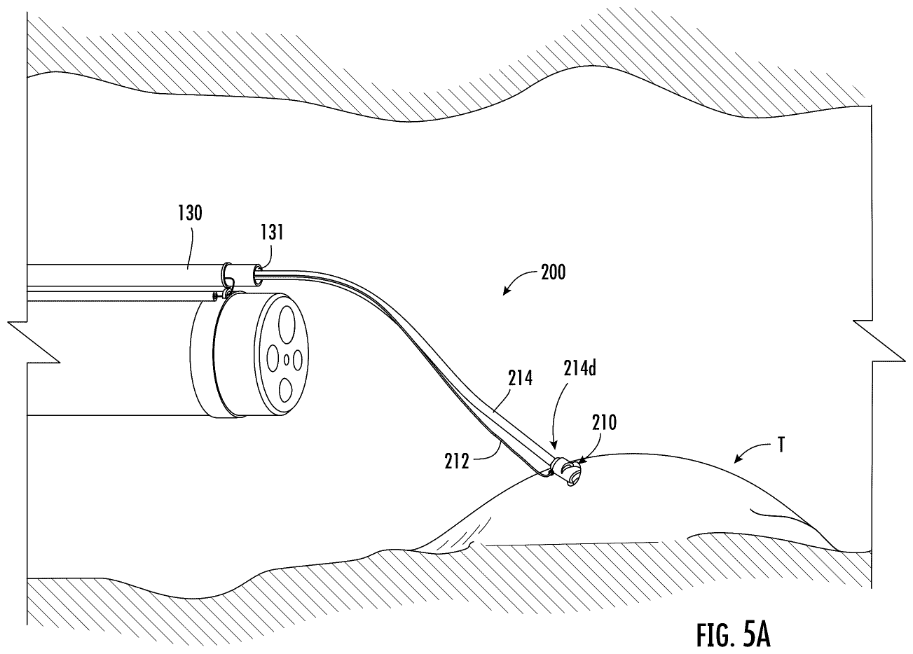

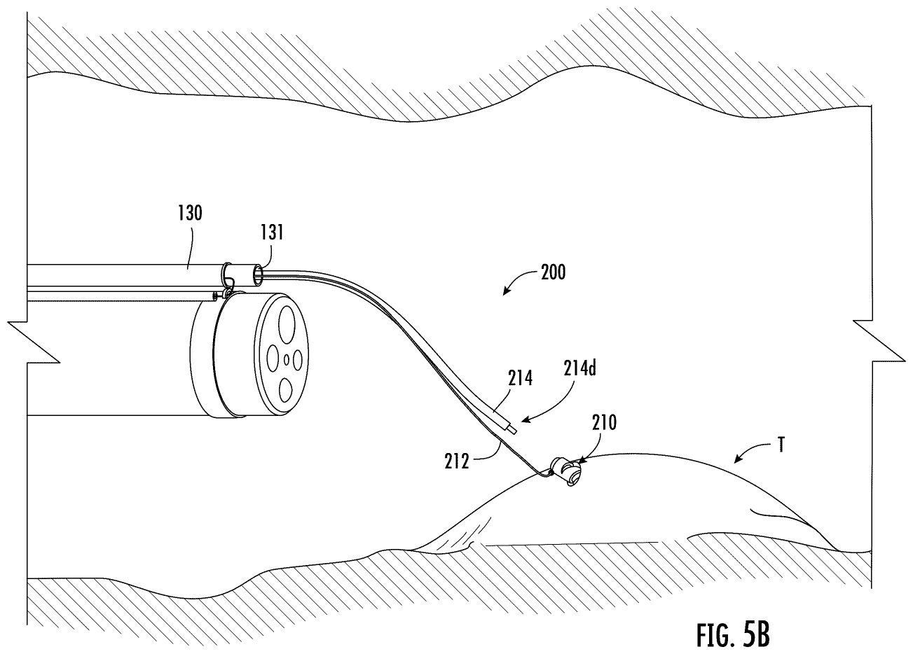

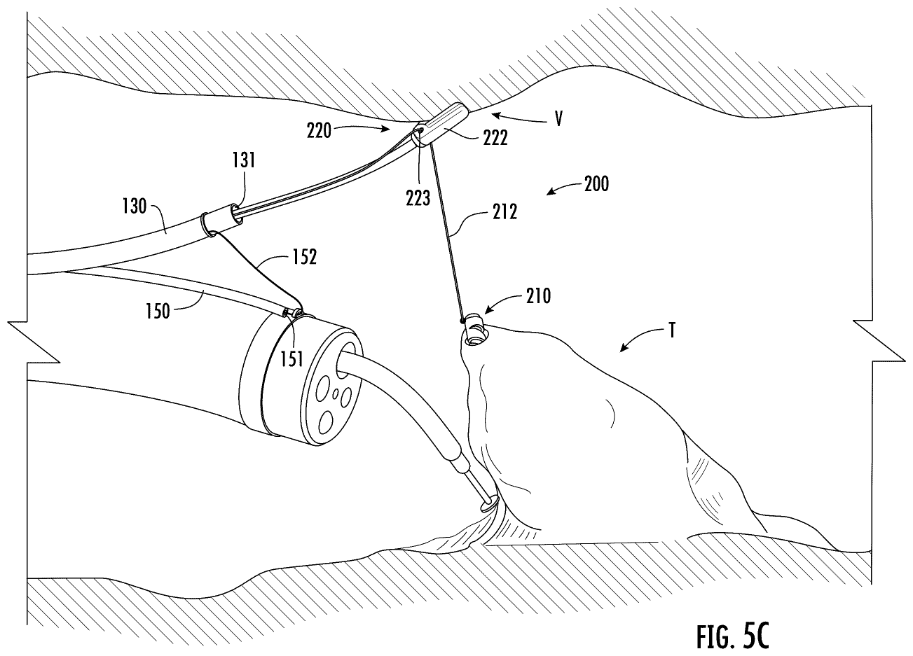

In some aspects, the tissue-lifting tissue-engagement element 210 of a tissue traction system 200 is deployed with an elongate control element 214 which is not operably associated with the traction-adjusting tissue-engagement element 220, e.g., is not extended through a lumen through the elongate member 224 with which the traction-adjusting tissue-engagement element 220 is operably coupled. In such configuration, as illustrated in FIGS. 5A-5C, the tissue-lifting tissue-engagement element 210, operably coupled to the distal end 214d of elongate control element 214, need not be delivered and deployed with the traction-adjusting tissue-engagement element 220 (illustrated in FIG. 5C). The tissue-lifting tissue-engagement element 210 may be deployed and engaged with tissue in a manner as described above with reference to FIG. 4A, reference being made thereto for the sake of brevity and without intent to limit. Also similar to the above-described example of an embodiment, the elongate control element 214 may be removably (e.g., frangibly) coupled with the tissue-lifting tissue-engagement element 210 so that once the tissue-lifting tissue-engagement element 210 is securely engaged with tissue at the treatment site T, the elongate control element 214 may be separated from the tissue-lifting tissue-engagement element 210, as illustrated in FIG. 5B. The traction-adjusting tissue-engagement element 220 may be delivered and deployed, as illustrated in FIG. 5C, to engage the traction-adjusting tissue-engagement element 220 with tissue at a vector-changing location V, such as in a manner described above with reference to FIG. 2C and FIG. 2D, reference being made to the above descriptions thereof for the sake of brevity and without intent to limit. The elongate element 212 of the tissue traction system 200 is operably associated with the tissue-lifting tissue-engagement element 210 during engagement of the tissue-lifting tissue-engagement element 210 with tissue, and may be operably associated with the traction-adjusting tissue-engagement element 220 in any of a variety of manners such as described above. For instance, and without intent to limit, as illustrated in FIG. 5C, the elongate element 212 may be extended through an aperture 223 defined through the traction-adjusting tissue-engagement element 220 so that the traction-adjusting tissue-engagement element 220 may function as a pulley when engaged with tissue at a vector-changing location V, such as illustrated in FIG. 5C. In some aspects, the elongate element 212 is operably associated with the traction-adjusting tissue-engagement element 220 and/or the tissue-lifting tissue-engagement element 210 so that relative movement (e.g., rotational movement) of the tissue-engagement elements 210, 220 does not result in entanglement, twisting, etc., of the elongate element 212. For instance, any of the above-described manners of coupling an elongate element with respect to a tissue-engagement element (e.g., such as, without limitation, described with respect to FIG. 4A and FIG. 4B), may be used with the example of an embodiment illustrated in FIGS. 5A-5C, reference being made to the above descriptions for the sake of brevity and without intent to limit.

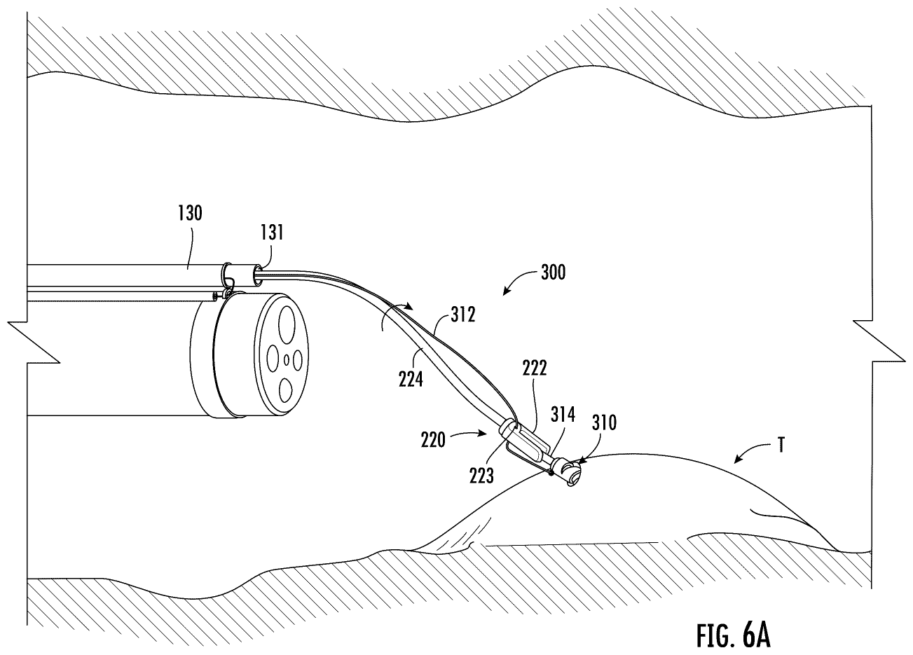

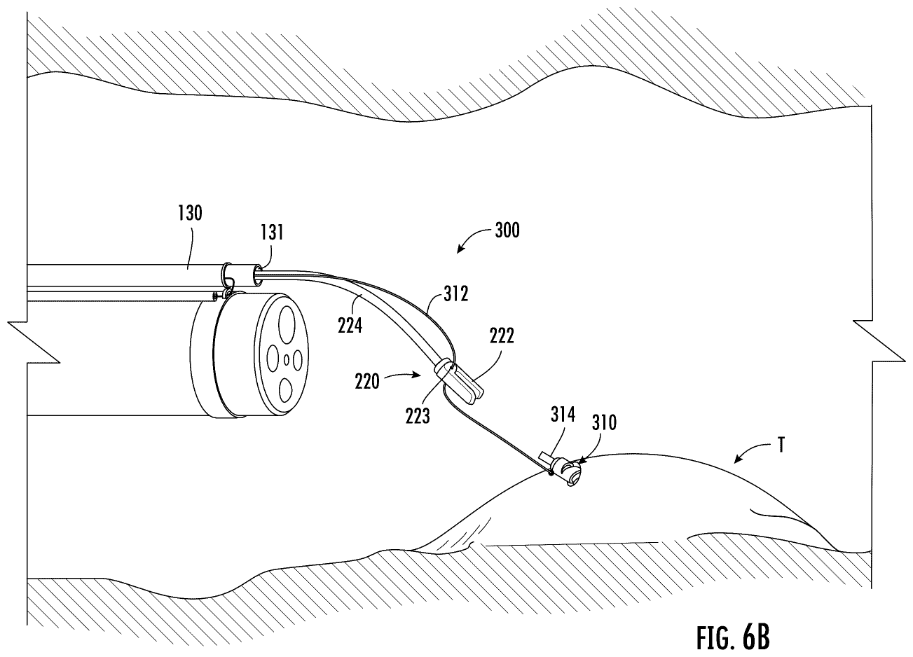

In some aspects, instead of the tissue-lifting tissue-engagement element being deployed and engaged with tissue with the use of an elongate control element, the traction-adjusting tissue-engagement element of a tissue traction system 300 may be utilized to deploy and engage the tissue-lifting tissue-engagement element with tissue, such as illustrated in FIG. 6A and FIG. 6B. More particularly, the tissue-lifting tissue-engagement element 310 of the tissue traction system 300 illustrated in FIG. 6A is delivered to the treatment site T grasped by the traction-adjusting tissue-engagement element 220. In the illustrated example of an embodiment, the traction-adjusting tissue-engagement element 220 is in the form of a tissue grasper with a pair of jaws 222, such as similar to the above-described traction-adjusting tissue-engagement elements. The jaws 222 of the traction-adjusting tissue-engagement element 220 are illustrated in FIG. 6A as grasping the tissue-lifting tissue-engagement element 310. In some aspects, the tissue-lifting tissue-engagement element 310 may include an extension 314 which is grasped by the jaws 222 of the traction-adjusting tissue-engagement element 220. In the illustrated example of an embodiment, the tissue-lifting tissue-engagement element 310 is in the form of a helical tissue-engagement element, and the traction-adjusting tissue-engagement element 220 may be rotated to rotationally advance the tissue-lifting tissue-engagement element 310 into tissue to be securely engaged therewith. However, it will be appreciated that other configurations of a tissue-lifting tissue-engagement element 310 may be delivered and deployed with the assistance of the traction-adjusting tissue-engagement element 320 of the tissue traction system 300. It will be appreciated that the elongate element 312 may extend from the tissue-lifting tissue-engagement element 310 in a manner which does not interfere with deployment and engagement of the tissue-lifting tissue-engagement element 310 with tissue, as may be appreciated by those of ordinary skill in the art. For instance, any of the above-described manners of coupling an elongate element with respect to a tissue-engagement element (e.g., such as, without limitation, described with respect to FIG. 4A and FIG. 4B), may be used with the example of an embodiment illustrated in FIG. 6A and FIG. 6B, reference being made to the above descriptions for the sake of brevity and without intent to limit. The elongate element 312 may be operably associated with the traction-adjusting tissue-engagement element 220 by extending through an aperture 223 formed therethrough (such as illustrated in FIG. 6A and FIG. 6B). Alternatively, the elongate element 312 may be operably associated with the traction-adjusting tissue-engagement element 220 by extending through a lumen defined through the elongate member 224 with which the traction-adjusting tissue-engagement element 220 is delivered, such as described above with reference to FIG. 3, reference being made thereto for the sake of brevity and without intent to limit.

It will be appreciated that principles of the present disclosure may be applied to deployment of additional tissue-engagement elements with respect to tissue at a treatment site and different vector-changing location. It will further be appreciated that the order of deployment of the tissue-engagement elements of a tissue traction system of the present disclosure need not be as described with respect to FIGS. 2A-2H. For instance, in some aspects, the tissue-lifting tissue-engagement element may be deployed after a traction-adjusting tissue-engagement element is deployed.

It will further be appreciated that although the present disclosure is described with reference to transluminal/endoscopic use, principles of the present disclosure are equally applicable to other methods of use. The traction devices and systems described herein may be used with a variety of medical devices for navigating body lumens, including, for example, catheters, insertion tubes, overtubes, endoscopes, laparoscopes, ureteroscopes, bronchoscopes, colonoscopes, arthroscopes, cystoscopes, hysteroscopes, etc. The disclosed medical devices and systems may also be inserted via access points and approaches other than through natural orifices (e.g., endoscopically), such as percutaneously, laparoscopically, or combinations thereof.

It is to be understood by one of ordinary skill in the art that the present discussion is a description of illustrative examples of embodiments only, and is not intended as limiting the broader aspects of the present disclosure. Although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like. Various further benefits of the various aspects, features, components, and structures of a tissue traction system and associated devices and methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A tissue traction system for transluminal delivery into a patient's body to apply traction to tissue at a treatment site within the patient's body, said tissue traction system comprising:
a tissue-lifting tissue-engagement element;
a traction-adjusting tissue-engagement element; and
an elongate element having a distal end coupled to said tissue-lifting tissue-engagement element, and extending from said tissue-lifting tissue-engagement element to be operably associated with said traction-adjusting tissue-engagement element for relative movement with respect thereto, said elongate element extending proximally and transluminally to a proximal end controllable from outside the patient;
wherein traction applied to said tissue-lifting tissue-engagement element by said elongate element is adjustable within the patient's body by pulling on the elongate element from outside the patient to move the elongate element with respect to the traction-adjusting tissue-engagement element as a pulley to apply traction to the tissue-lifting tissue-engagement element.

2. The tissue traction system of claim 1, wherein said elongate element extends from said tissue-lifting tissue-engagement element, through a portion of said traction-adjusting tissue-engagement element, and proximally to the proximal end outside the patient's body.

3. The tissue traction system of claim 2, wherein said traction-adjusting tissue-engagement element defines an aperture therethrough and said elongate element extends through the aperture.

4. The tissue traction system of claim 2, wherein said traction-adjusting tissue-engagement element is operably coupled to an elongate member defining a lumen therethrough, said elongate element extending through the lumen to outside the patient's body.

5. The tissue traction system of claim 1, further comprising a tubular elongate member defining a lumen through which said tissue-lifting tissue-engagement element and said traction-adjusting tissue-engagement element are delivered.

6. The tissue traction system of claim 5, wherein said tubular elongate member is delivered with a medical delivery device defining a working channel through which a medical instrument is deliverable into the patient.

7. The tissue traction system of claim 6, wherein said tubular elongate member is laterally separable from the medical delivery device to allow independent movement of the medical delivery device while the traction-adjusting tissue-engagement element is engaged with tissue.

8. The tissue traction system of claim 1, further comprising an elongate control element having a distal end coupled to said tissue-lifting tissue-engagement element and operable to engage and deploy the tissue-lifting tissue-engagement element with tissue.

9. The tissue traction system of claim 8, wherein said elongate control element is rotatable to rotationally engage the tissue-lifting tissue-engagement element with tissue, and is frangibly coupled with the tissue-lifting tissue-engagement element to be separated therefrom after the tissue-lifting tissue-engagement element is deployed with respect to tissue.

10. The tissue traction system of claim 1, wherein the traction-adjusting tissue-engagement element is engageable with the tissue-lifting tissue-engagement element and movable to engage and deploy the tissue-lifting tissue-engagement element with tissue.

11. The tissue traction system of claim 1, wherein the traction-adjusting tissue-engagement element is configured to be engageable with tissue spaced apart from the treatment site, releasable from the engaged tissue, and movable to another location spaced apart from the treatment site and engaged with tissue at the other location.

12. A tissue traction system for transluminal delivery into a patient's body to apply traction to tissue at a treatment site within the patient's body, said tissue traction system comprising:

13. a tissue-lifting tissue-engagement element;

14. a traction-adjusting tissue-engagement element;

15. an elongate element having a first end coupled to said tissue-lifting tissue-engagement element, and extending from said tissue-lifting tissue-engagement element to be operably associated with said traction-adjusting tissue-engagement element; and

16. an elongate member operably coupled with said traction-adjusting tissue-engagement element to move said traction-adjusting tissue-engagement element to be engaged with tissue at a first location, to move said traction-adjusting tissue-engagement element from the first location to a second location, and to move said traction-adjusting tissue-engagement element to be engaged with tissue at the second location to adjust the traction force vector applied by said elongate element to the tissue at the treatment site via said tissue-lifting tissue-engagement element.

17. The tissue traction system of claim 12, wherein said elongate element is operably associated with said traction-adjusting tissue-engagement element to be translatable with respect to said traction-adjusting tissue-engagement element to adjust the traction force vector applied by said elongate element to said tissue-lifting tissue-engagement element.

18. The tissue traction system of claim 12, wherein said traction-adjusting tissue-engagement element comprises a pair of jaws shiftable between a tissue-receiving configuration in which said jaws are spaced apart to receive tissue therebetween or to release tissue, and a tissue-grasping configuration in which said jaws grasp tissue therebetween, said jaws being controllable from outside the patient's body to engage the traction-adjusting tissue-engagement element with tissue at the first location, to release tissue at the first location, and to engage tissue at the second location.

19. The tissue traction system of claim 14, wherein:

20. said tissue-lifting tissue-engagement element is helical and said jaws of said traction-adjusting tissue-engagement element are engageable with said tissue-lifting tissue-engagement element to rotationally engage said tissue-lifting tissue-engagement element with tissue to engage tissue; and

21. said tissue-lifting tissue-engagement element includes an extension graspable by said jaws of said traction-adjusting tissue-engagement element to rotationally advance the tissue-lifting tissue-engagement element into tissue.

22. A method of applying traction to tissue within a patient, said method comprising:

23. transluminally advancing a tissue-lifting tissue-engagement element and a traction-adjusting tissue-engagement element with an elongate element extending therebetween to a treatment site within a patient;

24. engaging the tissue-lifting tissue-engagement element with tissue at the treatment site;

25. engaging the traction-adjusting tissue-engagement element with tissue at a first location spaced apart from the treatment site;

26. extending the elongate element from the tissue-lifting tissue-engagement element to the traction-adjusting tissue-engagement element and proximally to outside the patient; and

27. proximally pulling on the elongate element from outside the patient to apply traction to tissue at the treatment site via the elongate element and the tissue-lifting tissue-engagement element.

28. The method of claim 16, further comprising moving the elongate element with respect to the traction-adjusting tissue-engagement element to adjust the traction force vector along the elongate element.

29. The method of claim 17, further comprising extending the elongate element from the tissue-lifting tissue-engagement element to through an aperture defined through the traction-adjusting tissue-engagement element and then proximally out of the patient's body.

30. The method of claim 17, further comprising advancing the traction-adjusting tissue-engagement element into the patient coupled to a distal end of an elongate member, and extending the elongate element from the tissue-lifting tissue-engagement element and into a lumen defined through the elongate member and proximally out of the patient's body.

31. The method of claim 16, further comprising disengaging the traction-adjusting tissue-engagement element from the first location, moving the traction-adjusting tissue-engagement element to a second location spaced apart from the target tissue, and engaging the traction-adjusting tissue-engagement element with tissue at the second location to adjust the traction force vector applied to the elongate element by pulling on the elongate element from outside the patient.
